(19) United States
(12) Patent Application Publication
(10) Pub. No.: US 2011/0301490 A1
(43) Pub. Date: Dec. 8, 2011

(54) DEVICE AND METHOD FOR DETECTING ELECTRIC POTENTIALS ON THE HUMAN OR ANIMAL BODY

(75) Inventors: Martin Mucke, Hilden (DE); Matthias Fahle, Munchen (DE)

(73) Assignee: UP MANAGEMENT GMBH, Munchen (DE)

(21) Appl. No.: 13/133,469

(22) PCT Filed: Dec. 2, 2009
(86) PCT No.: PCT/EP2009/008595

§ 371 Date: Aug. 29, 2011

(30) Foreign Application Priority Data
Dec. 12, 2008 (EP) 10 2008 062 018.1

(51) Int. Cl. A61B 5/053

(52) U.S. Cl. 600/547

(57) ABSTRACT

The invention relates to a device and method for detecting electrical potentials on the human or animal body, with a body-side arrangement of electrodes (3) and a body-side management unit (4). According to the invention, the conductive pathways (19) between electrodes (3) and management unit (4) include at least in certain areas electrically conductive liquids (17). The pathways (19) are preferably designed as fluid-filled pad segments (16). The invention also relates to a device and method of the aforementioned kind with a body-side arrangement of electrodes (3) and a preferably body-side management unit (4) and/or a preferably remote processing unit (10), which communicate with each other by means of radio transmission (8) using at least one transmitter (7) and at least one receiver (9). It is contemplated that the means of radio transmission (8) preferably comprise transmitter (7) and receiver (9) according to the Zig Bee, Wibree, Bluetooth or ULP standard. The invention also relates to a clothing-like supporting means for such a device.

The invention relates to a device for detecting electrical potentials on the human or animal body, with a body-side arrangement of electrodes and a body-side management unit. The invention also relates to a device for detecting electrical potentials on the human or animal body, with a body-side arrangement of electrodes and a preferably body-side management unit and/or a preferably remote processing unit, communicating with each other, and to a clothing-like supporting means and a method for detecting electrical potentials on the human or animal body.

### PRIOR ART

Device and method of the aforementioned type are known from the published patent application DE4215549A1. In this document, a telemetric transmitter is disclosed, whose transmission electronics, being arranged in-between plastic layers, and designed with electrodes as a component and communicates with these via a conductive plastic layer. The components including a battery are inserted water-tight into a belt, which can be worn around the chest. The data detected from the electrode, such as ECG or heart rate of the user is transmitted wirelessly to the receiver using a magnetic near field. The receiver for example, can be worn on a bracelet.

Another generic device for mobile surveillance of the cardiac function is described in the published patent application DE102005060985A1. The device has several electrodes which are connected to the breast and that are wirelessly connected to a body-side arranged management unit. The electrodes are equipped with Bluetooth transmission devices and power supplies that enable the transmission of electrical signals conducted to the management unit.

The management unit itself has a signal processing device, in particular an A/D converter, and a memory device. It is also equipped with analysis software to identify critical heart functions. Via a USB or Bluetooth interface, the data recorded in the memory device can also be transmitted to another medium.

Another embodiment disclosed in this publication relates to the data transfer between electrodes and management unit via electrical conductors that are arranged in a garment. This garment is worn by the patient during the test rather than conventional clothes. Thus, the freedom of movement of the patient is not restricted. Additionally, the burden of extra weight is quite low.

Another device for wireless recording and remote monitoring of electrical potentials is disclosed in the published patent document DE102004020515A1. Attached on the patient, in particular an animal, is a miniaturized data acquisition means in the form of a small, battery-operated device, which detects analogue signals from the electrodes which are connected with the shortest possible cables. Battery operated device performs the signal digitization, error correction and data processing. For this purpose, through appropriate algorithms and digital signal filters any interference and noise components are eliminated, so that the transmitter has a full EMG or ECG trace. Over a digital wireless connection path, preferably using Bluetooth technology, this data is transmitted to the receiver, which is arranged a sufficient distance from the patient and connected to a computer. The patient data is then merged and visualized.

The transmission according to the Bluetooth standard has the disadvantage of high energy consumption and a relatively long period of time is required to make the wireless connection between electrodes and management unit.

In European Patent EP1331878B1 a pacemaker is also described, which is equipped with a device for conducting electrical potential and its wireless transmission to a body remote monitoring station. The spiral-shaped electrodes are placed subcutaneously and are isolated against direct contact with body tissue and moving body fluids. The electrodes communicate by cable with a body-side management unit, which is arranged in a hermetically sealed housing and which has a device for signal processing. The connection made by radio link between the monitoring station and management unit allows bi-directional communication between the body side and the remote components of the device.

A complex wiring between the electrodes and the management unit must be provided here as well.

### Object

The invention has the object of developing devices and procedures of this genus so that they provide a high application and wearing comfort, that have low energy consumption, and enable reliable and highly responsive data exchange between the electrodes, the management unit and/or the processing unit.

### Solution

The object is solved in a first embodiment of the invention for a device of the aforementioned type in that the pathways between electrodes and management unit include at least in certain areas electrically conductive liquids. In a departure from the prior art, the communication is not provided over an uncomfortable to handle and (with respect to the wearability) problematic wiring, but can be made through any liquid depots, which are electrically isolated from each other according to the number of required pathways.

The inventive device may, for example be used for the conduction of an electrocardiogram (ECG) signal, but also, more generally, for use in the acquisition of brain waves (EEG) or other electrical potentials (for example, electromyography EMG).

The management unit comprises the advantage of a transmitter that communicates wirelessly with the receiver of a processing unit. This makes it possible to arrange the relatively large processing unit, which typically has data storage, display and printing devices, remote from the body and therefore allowing substantial freedom of movement for the patient.

Particularly preferred is an embodiment of the invention in which at least one, preferably several electrodes are arranged with the management unit in a body-side unit. This unit is applied to the patient as a whole, so a complex component-wise arrangement and faults associated therewith are eliminated.

Preferably, the unit of electrodes and management unit is formed in the form of a preferably flat pad. A pad consisting of a soft pad cover and pad filling has a great ability to adapt to the body surface of the patient and provides high wearing comfort.

In respect of a simple application, the unit is advantageously angularly formed, preferably also curved or executed with rounded angles, whereas at each leg end and preferably also in the transition area between the leg ends one electrode is arranged.

The handling of the unit is also preferably facilitated by the fact that the unit is equipped with an adhering outer surface by which the unit can be attached to the body surface. Before using the unit, the adhering surface may be protected by a silicone-coated paper or the like from unwanted contact.

According to a particularly advantageous embodiment of the invention, the pathways are formed as fluid-filled pad segments which form at least part of the pad filling. The pad segments can, for example, be honeycomb-like, but may also be formed with a substantially elongated extension.

The cover of the pad is preferably made of an electrical insulator, especially a thermoplastic synthetic material. Without further addition, the electrical separation between the pathway forming liquid deposits is thus provided by the pad cover (including, if intended dividers provided in the pad cover).

The liquid used for signal transmission is advantageously an aqueous electrolyte, which can be cheaply produced and has a sufficient conductivity.

The liquid also preferably shows a gel-like consistency, by which the wearing comfort with regard to haptics and noise generation is improved.

A liquid, particularly suitable for carrying out the invention contains 0.65 to 0.75 wt %, preferably about 0.71 wt % demineralized or distilled water, 0.10 to 0.20 wt %, preferably about 0.14 wt % sodium chloride, 0.03 to 0.07 wt %, preferably about 0.05 wt % hydroxyethyl cellulose and 0.07 to 0.13 wt %, preferably about 0.10 wt % propylene glycol. To the thus obtained gel-like liquid a dye can be additionally added. The gel is preferably prepared by mixing powdered sodium chloride with likewise powdered hydroxyethylen cellulose, subsequent addition of liquid propylene glycol and filling the mixture with water under stirring.

The object underlying the invention is further achieved in that the communication takes place wirelessly using means for radio transmission with a transmitter and receiver. For radio transmission standards such as W-LAN or Bluetooth can be used. However, it is preferred that the means for radio transmission of patient data comprise transmitter and receiver according to the ZigBee radio network standard. ZigBee is a wireless radio standard, which is based on the IEEE802.15.4 standard and particularly appropriate for data transmission to short distances between 10 and 100 meters. The features in the frequency ranges 863.3 MHz/2.46 GHz (Europe) and 915 MHz/2.46 GHz (USA) of the ZigBee wireless radio network standard is characterized by a very low energy consumption of the terminals and a stable and secure wireless connection with a transmission rate of 20-250 kbit/s. The reaction times are so low that real-time applications are possible. The ZigBee wireless transmission may be either from the electrodes to the management unit and preferably takes place between the body-side management unit and the typically remote control unit.

The standard IEE802.15.4 provides with very basic and cost efficient-RFD (Reduced Function Devices) and FFD (Full Function Devices) two classes of devices of different complexity. An FFD can communicate with other FFDs and with RFDs while an RFD can be connected to only one FFD. The software implementation is possible with an 8-bit microcontroller. With the two ZigBee device classes different network topologies can be constructed, such as a star topology in which RFDs or FFDs are connected to a FFD acting as a PAN-Coordinator. Alternatively, a peer-to-peer topology is conceivable providing a point-to-point communication between the devices which are located within the radio range.

When setting up wireless networks in devices for the conduction of electrical potentials for medical purposes, differentiation between on the one hand the sensor nodes which receive and transmit the data to a central node, and on the other hand the actuator nodes which are addressed by a central node is required.

The electrodes are preferably connected in operative connection to a body-side measurement transducer (A/D converter), which in turn communicates with the body-side transmitter. Here, transmitter and/or transducer are preferably designed as single-use chipset.

The management unit may however in principle be provided both for single and for multiple use. In the latter case it is preferably provided, that the management unit is set up through a connector on the pad case and thus electrically connects with the conductive liquid.

The reception of data transmitted can be effected by a receiver with a PCMCIA card or a viewer, which in addition to the ZigBee receiver chip, be also barcode or RFID readers integrated to allow the pairing.

Furthermore, the transmission according to the Wibree standard or the Upper Layer Protocol (ULP) is also possible. The Wibree standard is a competitor to the industry-standard Bluetooth and transmits in the range of 2.4 GHz, but with significantly reduced power input and range. The transmission rate is 1 Mbit/s. ULP is an application of the Socket Direct Protocol (SDP).

With regard to a clothing-like supporting means for a device for detecting electrical potentials on the human or animal body, especially according to one of the previously described embodiments, the object is achieved in that the supporting means is provided with means to adapt to the body size. The concept of body size covers all dimensions of the human or animal body, for example, also the abdomen, arm or neck circumference, shoulder width and height of the upper body. Thereby, the supporting means can be worn by patients of very different body sizes and without adversely affecting the quality of the measurement or wearing comfort. Especially preferably the supporting means is constructed like a vest and has a variable adjustment means for width, length, neck size, arm length and/or shoulder width.

Further, to achieve the object the measures mentioned in the method claims are suitable.

### FIGURES

The figures represent an example and schematically different embodiments of the invention;

They show:

FIG. 1 shows the basic structure of an inventive device using the example of an electrocardiogram (ECG);

FIG. 2 shows a top view of such a device,

FIG. 3 shows a longitudinal section through the device of FIG. 2;

FIG. 4 shows a top view of another inventive device;

FIG. 5 shows a liquid-filled pathway according to another embodiment of the invention;

FIG. 6 shows the electrodes communicating with the supply unit over a radio link,

FIG. 7 shows a carrying device for an electrode array.

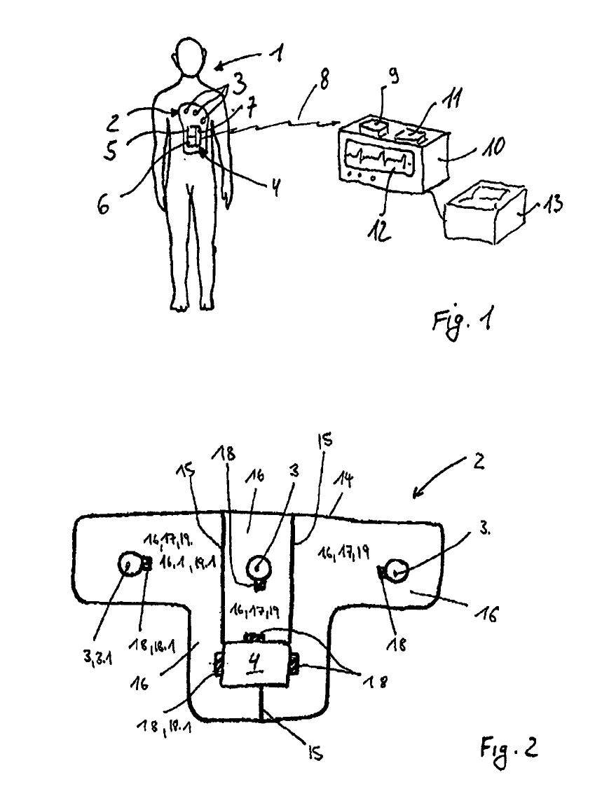

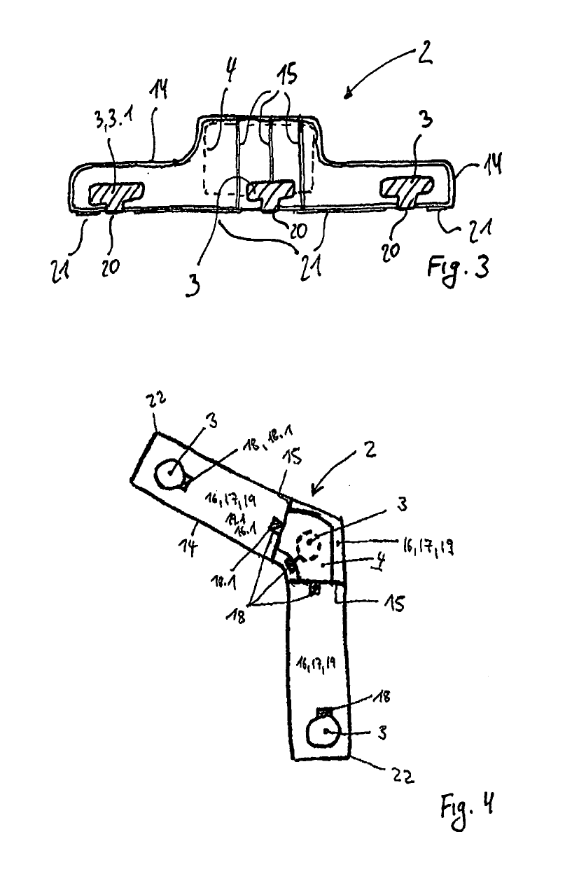

As shown in FIG. 1, the patient 1 is wearing a pad-like unit 2 on the chest area, which houses a number of the body surface overlying electrodes 3 which are connected to a management unit 4. The management unit 4 comprises a measurement transducer (A/D converter) 5, a power supply unit 6 and a transmitter 7, which works on ZigBee wireless radio network standard.

The body-side unit 2 communicates by radio transmission 8 to the receiver 9 of the processing unit 10 being remotely disposed, which also includes a data memory 11 and a display 12 for graphical display of a measured curve. Furthermore, the processing unit 10 is equipped with a printing device 13 for printing the measured curve.

The flat, pad-shaped unit 2 consists of an electrically insulating pad cover 14 which surrounds on the one hand the unit 2 completely and otherwise structures the unit 2 by bars 15 into a plurality of electrically separate pad segments 16. Pad cover 14 and bars 15 can be cut of a film of a flexible, thermoplastic synthetic material, particularly polyethylene or a polyethylene/polypropylene copolymer and then be welded together. Each pad segment 16 is filled with an electrically conductive gel-like fluid 17. The unit 2 is provided, including electrode 3 and the complete management unit 4 as a disposable item, i.e. it is to be discarded after single use.

Both the electrodes 3 as well as the management unit 4 are provided with electrical contacts 18 which protrude into the liquid 17. In each case, an electrical contact 18 of an electrode 3 is via a liquid 17 filled pad segment 16 in electrically conductive connection with an associated electrical contact 18 of the management unit 4. For example, the contact 18.1 of the electrode 3.1 communicates via the pathway 19.1 formed by pad segment 16.1 forming with the contact 18.1′ of the management unit 4.

The electrodes 3 penetrate the pad cover 14 locally and are provided in these regions 20 for direct contact with the body surface of patient 1. It is understood that the openings be sealed, that leakage of liquid 17 located in the pad segments 16 is excluded. The adjacent outer surfaces 21 of the pad cover 14 are self-adhesive and used to arrange the unit 2 to the body of the patient 1.

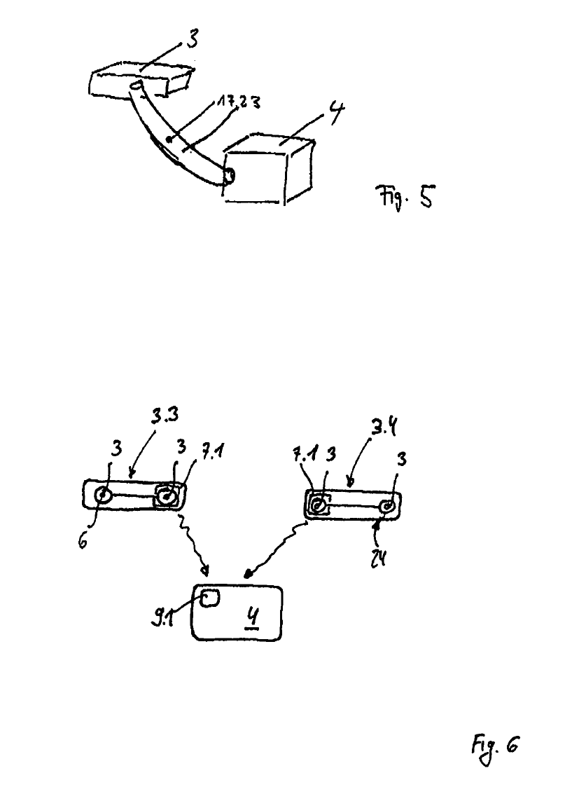

In the embodiment of FIG. 4, the unit 4 according to the desired electrode arrangement is formed angularly where at each leg end 22 and within the angle of the electrode array one electrode 3 is arranged. The center electrode 3 is located underneath the management unit 4. Here too, the electrical connection between the electrodes 3 and the management unit 4 is achieved via liquid 17. So again, the contact 18.1 of electrode 3.1 is connected over the electrical pathway 19.1 forming, with liquid 17 filled pad segment 16.1 with the contact 18.1′ of the management unit 4. The other contacts 18 communicate accordingly.

FIG. 5 illustrates an inventive device, in which the electrode 3 and the management unit 4, are not forming an unit, but communicate electrically as separate components through a tube line 23 filled with conductive liquid 17. In this case, to determine the potential differences any number of electrodes 3 can be connected via associated tube lines 23 to the management unit 4.

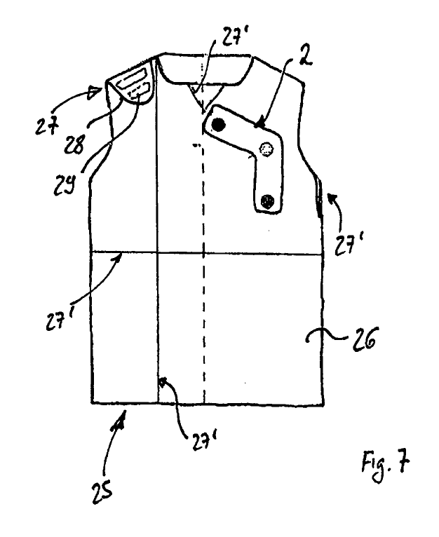

As shown in FIG. 6, the present invention includes the wireless communication of electrode clusters 3.3, 3.4 from at least two electrodes 3 and a transmitter 7.1, to the receiver 9.1 of the management unit 4, in particular by radio transmission 8 based on the ZigBee wireless radio network standard. In this case, each electrode 3 is equipped with a power supply unit 6 in the form of a button battery. The outer surfaces 24 of the electrode cluster 3.3, 3.4 also have RFID tags which, for the purpose of setting-up the network, establish a binding for the individual electrode clusters 3.3, 3.4.

As is shown in FIG. 7 support means 25 consists of a vest-like garment 26 which is provided in the chest area with unit 2 according to the embodiment of FIG. 4. The garment 26 is fitted with adjustment means 27 for example in the shoulder area with a cross strap 28 gripping from behind over the shoulder, which are fixed by hook and loop fasteners 29 in different positions on the front of the garment 26, to match the body size of patients. Similar adjustment means 27′ for adjusting the length, width and the neck and arm width are provided.

### REFERENCE SIGN LIST

1 Patient
2 Unit
3, 3.1, 3.2 Electrode
3.3, 3.4 Electrode Cluster
4 Management Unit
5 Measurement Transducer
6 Power supply unit
7, 7.1 Transmitter
8 Radio Transmission
9, 9.1 Receiver
10 Processing unit
11 Data Memory
12 Display
13 Printing device
14 Pad Cover
15 Bar
16, 16.1 Pad Segment
17 Liquid
18, 18.1, 18.1′ Contact
19, 19.1 Pathway
20 Regions (for direct contact)
21 Outer Surface (Self adhesive of pad cover)
22 Leg End
23 Tube Line
24 Outer Surface (Electrode)
25 Supporting means
26 Garment
27, 27′ Adjustment means
28 Shoulder
29 Hook and Loop Fastener

1. Device for detecting electrical potentials on a human or animal body with a body-side arrangement of electrodes and a body-side management unit, characterized in that at least one pathways between the electrodes and the management unit comprises, at least in certain areas, an electrically conductive liquid.

2. The device of claim 1, wherein the management unit comprises a transmitter, which communicates wirelessly with a receiver of a processing unit.

3. The device of claim 1, wherein at least one electrode is arranged with the management unit in a body-side unit.

4. The device of claim 3, wherein the body-side unit is formed from electrodes and the management unit in the form of a flat pad.

5. The device of claim 3, wherein the body-side unit is formed angularly, wherein at each leg end and in the transition area between the legs an electrode is arranged.

6. The device according to claim 3, wherein the body-side unit is provided with an adhesive outer surface.

7. The device according to claim 4, characterized in that the conductive pathways are formed as liquid-filled pad segments.

8. The device according to claim 4, characterized in that the pad cover is manufactured from an electrical insulator, the insulator comprising a thermoplastic synthetic material.

9. The device according to claim 1, wherein the liquid consists of an aqueous electrolyte.

10. The device of claim 1, wherein the liquid has a gel-like consistency.

11. The device of claim 10, wherein the liquid contains about 0.65 to 0.75 wt-%, preferably about 0.71 wt % demineralized or distilled water, about 0.10 to 0.20 wt-% , preferably about 0.14 wt % sodium chloride, about 0.03 to 0.07 wt-%, preferably about 0.05 wt % hydroxyethyl cellulose and about 0.07 to 0.13 wt-%, preferably about 0.10 wt % propylene glycol.

12. A device for detecting an electrical potential on a human or animal body, having a body-side arrangement of electrodes and a body-side management unit and/or a processing unit that communicate with each other, according to claim 1, wherein the communication means uses wireless radio transmission with a transmitter, and a receiver based on the ZigBee wireless networking standard.

13. The device according to claim 12, wherein the electrodes are in operative connection with a body-side converter, which communicates in turn with the body-side transmitter.

14. The device of claim 12, characterized in that the transmitter and/or the measurement transducer are in the form of a single use chip.

15. Clothing-like supporting means for a device for detecting electrical potentials on the human or animal body, according to claim 1, wherein the supporting means is provided with means to adapt to the body size.

16. Supporting means according to claim 15, wherein the supporting means is vest-like and is due to adjusting means variable regarding width, length, neck size, arm length and/or shoulder width.

17. A method for signal transmission between electrodes and a management unit of a device for detecting electrical potentials on a human or an animal body, wherein the signals derived from the electrodes are transmitted to a management unit by means of an electrically conductive liquid.

18. A method for detecting electrical potentials on a human or an animal body and wireless transmission of data to a processing unit, in particular according to claim 17, wherein the transfer of data is communicated according to the ZigBee standard.

19. The method of claim 18, wherein the derived analog signals of the electrodes are digitized and then transferred to the processing unit.
